(19) United States
(12) Patent Application Publication
(10) Pub. No.: US 2025/0375110 A1
(43) Pub. Date: Dec. 11, 2025

(54) VENOUS DIAGNOSTIC CATHETER WITH FORCE MEASUREMENT AND DIAMETER INDICATION MECHANISMS

(71) Applicant: Vector Vascular, Inc., Solana Beach, CA (US)

(72) Inventors: Chris Hasselwander, Carlsbad, CA (US); Steven Howard, La Jolla, CA (US); Bradley Klos, Solana Beach, CA (US)

(21) Appl. No.: 19/229,975

(22) Filed: Jun. 5, 2025

Related U.S. Application Data
(60) Provisional application No. 63/657,608, filed on Jun. 7, 2024.

(51) Int. Cl. A61B 5/00; A61B 5/02; A61B 90/00

(52) U.S. Cl. CPC A61B 5/0048; A61B 5/02007; A61B 5/6858; A61B 5/6859; A61B 5/742; A61B 90/06; A61B 2090/064; A61B 2560/0223; A61B 2560/0462

(57) ABSTRACT

A catheter device may include a body. The body can include a handle body, an actuator, a force sensor, and a displacement sensor. The catheter device may include a scaffold. The scaffold can include a plurality of splines. Each of the plurality of splines can extend transversely and longitudinally along the body. The deployable scaffold can move between a first position to a second position and all positions in-between. The catheter device may further include a shaft assembly. The shaft assembly can include an outer shaft and inner shaft coupled to the body portion. In the first position, the scaffold can be in a collapsed state. In the second position, the scaffold can be in an expanded state. The force sensor and displacement sensor may be located in the body to measure force and/or displacement translated by the inner and outer shaft from the scaffold.

### INCORPORATION BY REFERENCE TO ANY PRIORITY APPLICATIONS

This application claims the priority benefit of U.S. Provisional Patent Application No. 63/657,608, filed on Jun. 7, 2024, and entitled “VENOUS STENTING CATHETER WITH FORCE MEASUREMENT AND INDICATION MECHANISMS”, the entire contents of which are hereby incorporated by reference herein and made part of this specification. Any and all applications for which a foreign or domestic priority claim is identified in the Application Data Sheet as filed with the present application are hereby incorporated by reference under 37 CFR 1.57.

### BACKGROUND

### Field

The present disclosure relates generally to intravascular medical devices, and more particularly to diagnostic catheter systems configured for temporary mechanical interaction with vascular structures to assess vessel luminal constrictive forces.

### Certain Related Art

Intravascular devices are medical tools designed to be placed within blood vessels. These tools can perform various purposes, including but not limited to administering fluids, medications, or providing access for monitoring hemodynamics. One example of intravascular devices include catheters.

### SUMMARY

Assessment of vascular lesions, choosing the right stent, and delivering said stent to a desired location can pose several concerns. For example, these steps may be costly, ineffective, and/or rely on inaccurate assumptions, resulting in complications in the body of a subject during and/or after a medical procedure. Thus, a need exists for improved devices, systems, and methods that can efficiently assess the mechanical properties of a vessel lumen at the site of suspected compression.

Other concerns, challenges, and/or desirable features of the intravascular devices are described below. For example, one challenge relates to assessments of vascular lesions, including non-thrombotic iliac vein lesions (NIVL), post-thrombotic syndrome (PTS), and other forms of venous compression or stenosis. Such assessments, when relying on imaging modalities and intravenous pressure measurements, may provide indirect and/or incomplete data regarding vessel compliance and mechanical constriction forces. Advantageously, the devices, systems, and methods of present disclosure can quantitatively assess the mechanical properties of a vessel lumen at the site of suspected compression, thereby providing more complete data.

Another challenge relates to unintended damages, alteration, and/or injury of surrounding biological tissues during a medical procedure that involves inserting a catheter device in a blood vessel. The devices, systems, and methods of present disclosure can temporarily and/or atraumatically (e.g., with reduced level of impact to the surrounding tissues) engage vascular structures. This engagement can expand the vessel lumen and measure the resulting forces, thereby providing diagnostic information to inform therapeutic decisions while reducing the level of unintended damage to the surrounding biological tissues. For example, the shaft assembly of the present disclosure can include one or more nested hypodermic tubes (also referred to as hypotubes) made from one or more materials (e.g., stainless steel) jacketed for an atraumatic surface finish (e.g., with one or more polymer). Advantageously, jacketing the outer surface of the shaft assembly can reduce any potential damage or discomfort to biological tissues during a medical procedure.

Moreover, it may be desirable for a catheter device to maintain the blood flow at the site of suspected compression while the catheter device is being inserted. The devices of the present disclosure can include an expandable scaffold with openings therebetween. Advantageously, this arrangement of scaffolds/openings can allow for continuous blood flow during the insertion and deployment of the scaffold, thereby reducing the risks and complications associated with the blockage of blood flow, or diagnostic inaccuracies resulting from the blockage of blood flow. Accordingly, in some embodiments, the devices of the present disclosure can be non-occlusive.

Another concern relates to the accuracy of measurement data provided by a catheter device, the complicated arrangement of sensors, and/or the position of sensors in the device. In some embodiments, the device of the present disclosure can be configured such that the sensor configuration is not sensitive to contact at the tip of the device, making the sensors of the catheter device more sensitive to forces applied to the scaffold. Advantageously, this can provide a more simplified design and/or facilitate obtaining more accurate data than if the sensor(s) were sensitive to contact at the tip of the device. In some embodiments, the device of the present disclosure can be configured such that sensors that measure the radial forces exerted by the vessel wall are not positioned on the stent scaffold. For example, the sensors can be positioned in the body portion of the catheter device and/or not directly in contact with the vessel wall. Advantageously, this arrangement of sensors can provide a more simplified design and/or facilitate obtaining more accurate data than if the sensor(s) were on the stent scaffold.

Additionally, the accuracy of measurement data provided by a catheter device may be related to the ability to measure the non-linear behavior of various features of the device in response to forces applied on the stent scaffold. In some embodiments, the device of the present disclosure includes an onboard processor and a calibration scheme that can account for the non-linear behavior of the scaffold, non-linear force sensitivity as the scaffold expands, and non-linear compensation for scaffold diameter under load, etc. For example, the devices of the present disclosure can include signal processing features that utilize a combination of analog signals, digital signals, and one or more calibration techniques. Advantageously, this allows the devices of the present disclosure to obtain measurement data (e.g., measurement of forces applied to the scaffold) that better reflect the behavior of said features during a medical procedure.

Another desirable feature of a catheter device may be the ability to be adaptable to accommodate different conditions. The catheter device of the present disclosure can, in some embodiments, include a scaffold and/or a shaft that can be adaptable to different conditions. For example, the length, strength, diameter, etc. of the scaffold and flexibility, buckle strength, etc. of the shaft can be changed as required to accommodate any application-specific needs. In one example, a stiffer scaffold can be used for higher force application(s). In another example, the geometry of the scaffold can be adjusted (e.g., the scaffold can open wider, etc.) to accommodate blood vessels with different openings. Advantageously, this allows the devices of the present disclosure to accommodate different applications, thereby reducing the costs and/or inefficiencies that would otherwise occur if a user (e.g., a medical professional) had to obtain a new device for different applications. Further, it may be desirable for the device disclosed herein to provide efficient and effective transfer of forces from the vessel wall to the sensor. Ineffective transfer of forces can result in inaccurate data being conveyed to the user. In some embodiments, the shaft assembly of the present disclosure is configured to resist buckling under expected operating forces of up to about 20 Newtons. Furthermore, the devices of the present disclosure can be configured to include one or more laser-cut patterns along a fractional distal length of the catheter portion of the device. Furthermore, the proximal portion of the shaft assembly can include a reduced number of cuts (e.g., include no cut) to facilitate force transmission onto one or more sensors in the handle. Advantageously, these features enable the device to balance flexibility (for deliverability) and axial stiffness (for force transfer).

Relatedly, it may be desirable to reduce/minimize the impact of friction on efficient and effective transfer of forces from the vessel wall to one or more sensors. The devices of the present disclosure utilize a variety of techniques, such as dimensional tuning, relative stiffness optimization, surface finishing, and/or use of low-friction coatings (e.g., PTFE) on inner shaft components and/or low-friction sleeves between shaft elements (e.g., PTFE, polyimide, nylon, etc.) to reduce the impact of friction on measured data. Accordingly, the shaft assembly of the present disclosure is configured to transmit torque efficiently to the distal end of the device while reducing friction and enabling controlled axial displacement of the scaffold.

Once inside the blood vessel, controlling the scaffold of a catheter device at the desired location may be a challenge. The devices of the present disclosure enable the expansion of the scaffold in a controlled and/or direct manner. Advantageously, this can facilitate a more accurate deployment of the stent scaffold at the desired location.

In some embodiments, the devices, systems, and methods of present disclosure can provide a diagnostic intravascular catheter system configured for temporary expansion of a vascular lumen and real-time measurement of radial forces exerted by the vessel wall during such expansion. The devices, systems, and methods of present disclosure can be configured for use within the venous system and be particularly suited for evaluation of venous compression syndromes, including NIVL and post-thrombotic conditions.

In some embodiments, the devices, systems, and methods of present disclosure can include a disposable, single-use catheter having an elongate shaft configured for advancement over a guidewire. The device can include a deployable scaffold structure at the distal end and a handle assembly incorporating actuation and feedback mechanisms. The scaffold can be configured to expand radially within the vessel lumen to temporarily displace the vessel walls outwardly without permanently altering vessel structure or compressing intraluminal lesions. In some embodiments, the devices of the present disclosure are not configured to deliver therapy but rather to provide diagnostic data regarding vessel compliance and resistance to expansion.

In some embodiments, the devices, systems, and methods of present disclosure can include integrated sensors configured to detect and display the expanded diameter of the scaffold and the radial forces encountered during vessel expansion, thereby providing the user with actionable diagnostic information regarding the nature and severity of vessel constriction.

In some embodiments, the devices, systems, and methods of present disclosure can include a diagnostic catheter system. The system can include an elongate tubular shaft having a proximal end, a distal end, and a central lumen configured to track over a guidewire. In some embodiments, the guidewire can be a 0.035″ guidewire. The system can include a distal scaffold assembly. The scaffold assembly can include a plurality of radially expandable elements formed from a superelastic material. In some embodiments, the superelastic material is nitinol. The scaffold can be configured to expand from a collapsed configuration to an expanded configuration within the vessel lumen. In some embodiments, the expanded configuration is in a range of clinically relevant sizes such as about 10 mm, about 20 mm, or any other value between about 10 mm and about 20 mm.

In some embodiments, the devices, systems, and methods of the present disclosure can include an actuation mechanism disposed within a handle assembly at the proximal end of the shaft. The actuation mechanism can include a user-operated actuator or rotary drive body (e.g., a knob) or other controls that can be configured to initiate and control the expansion of the scaffold assembly.

In some embodiments, the devices, systems, and methods of the present disclosure can include one or more integrated sensors configured to detect the expanded diameter of the scaffold and/or the radial force exerted by the vessel wall against the scaffold during expansion. In some embodiments, the system can detect a radial force of about 1N, 2N, 3N, 15N, 20N, smaller than about 1N, larger than about 20N, or any other value between about 1N and about 20N.

In some embodiments, the devices, systems, and methods of present disclosure can include one or more display modules integrated with or attached to the handle assembly. The display module can be configured to provide real-time feedback of the scaffold diameter and/or radial forces exerted on the scaffold.

In some embodiments, the devices, systems, and methods of present disclosure can include one or more batteries to provide power for self-contained operation.

In some embodiments, the devices, systems, and methods of present disclosure can be a medical device that can be deployed multiple times (e.g., one time, two times, three times, five times, etc.) within a single patient to provide one or more diagnosed sites within the vasculature with one or more stents.

In some embodiments, the devices, systems, and methods of present disclosure can include an electronic force sensor positioned proximal to the stent scaffold but distal to the flexible section of the catheter, thereby allowing similar sensing of vessels with higher tortuosity.

In some embodiments, the devices, systems, and methods of present disclosure can be configured for use over Intravenous Ultrasound (IVUS) catheter. For example, an inner diameter of the devices of the present disclosure can be tuned to allow a 3.5French (3.5F) IVUS catheter. In some embodiments, shaft material(s) between the distal and proximal ends of the stent scaffold can be tuned to be invisible to IVUS imaging.

In some embodiments, the devices, systems, and methods of present disclosure can include one or more blood pressure sensors positioned beneath the stent scaffold.

In some embodiments, the devices, systems, and methods of present disclosure can include a self-expanding mechanism with force feedback (non-driven). For example, the devices of the present disclosure can be configured to open to a certain diameter in the same manner as the intended stent to be deployed. This would allow a more direct simulation of stenting without the stent being necessarily adjustable, and/or be controlled.

In some embodiments, the devices, systems, and methods of present disclosure can include one or more support structures beneath the main stent scaffold, thereby increasing the radial strength of the scaffold.

In some embodiments, the devices of the present disclosure can include a scaffold, a shaft assembly, and a handle portion. The scaffold can serve as the primary compliant spring element for force sensing. The shaft can be designed with significantly higher (e.g., between about one time to about ten times more) axial stiffness than the scaffold to inhibit signal interference from shaft compliance. In some embodiments, the shaft assembly enters the handle portion as a nested hypotube assembly to maximize force transfer into the handle in non-tortuous portions of the anatomy. The handle portion can house a force sensing assembly situated between an actuated shaft and a fixed shaft. In some embodiments, the force sensing assembly is rigid. For example, the force sensor assembly is not included in the splines of the stent scaffold and thus does not flex (e.g., expand, contract, etc.) as the scaffold assembly moves from a collapsed position to an expanded position. The shaft assembly can be mechanically decoupled within the handle to isolate and measure the reactive force applied by the distal scaffold assembly. In some embodiments, the devices of the present disclosure can include anti-rotation features integrated at the shaft-handle interface to enable torque transmission during navigation without imparting rotational distortion during scaffold expansion. In some embodiments, the outer shaft is the actuated shaft, and the inner shaft is the fixed shaft. In some embodiments, the outer shaft is the fixed shaft, and the inner shaft is the actuated shaft. In some embodiments, the force sensor and/or displacement sensor can be positioned in at least one of: a) in the shaft, or b) partially in a shaft assembly and partially in the body portion 110, or c) partially in the scaffold 250, partially in the shaft assembly, and partially in the body portion 110. In some embodiments, the force sensor and/or displacement sensor are positioned at a distal end of the shaft assembly.

In some embodiments, the devices, systems, and methods of the present disclosure include a system. In one example, the system can include a catheter device and one or more devices used in a venous stenting procedure. For example, the system can include a catheter device disclosed herein and an IVUS catheter.

In some embodiment, the catheter technology of the present disclosure can assist in venous stenting procedures. The catheter can include foreshortening stent scaffold with mechanical force measurement means, which can be translated to a colored indicator window in the handle to allow for visualization of the force applied.

In some embodiments, the device of the present disclosure can include three mechanisms. For example, a radially expanding scaffold, a controlled deployment mechanism in handle, and a force feedback measurement. Radially expanding scaffold and/or struts which apply radial force can include one or more of a wall contact length with minimum 4 cm, expansion from 8.5French (8.5F) (2.83 mm) to 18 mm, known momentary expansion radius, and/or known momentary radial force exerted by scaffold.

In some embodiments, the distal expansion element can include foreshortening. For example, the devices disclosed herein can include driven foreshortening stent, driven foreshortening scaffold, and/or foreshortening scissor expansion mechanism. In some embodiments, the distal expansion element can include a self-expanding stent. For example, the devices disclosed herein can include a radial force. The radial force can be characterized. For example, a stent is “deployed” and recaptured during procedure.

In some embodiments, the scaffold expansion mechanism can include one or more pull wires to drive foreshortening scaffold, a push outer shaft relative to inner shaft, a pull outer shaft relative to outer shaft, a self expanding stent.

In some embodiments, known momentary expansion radius can include characterization of foreshortening versus scaffold diameter, wire indicator attached to inner surface of scaffold—translated to sight glass on handle, potentiometer on lever arm-anchored to shaft, lever attached to scaffold ID.

In some embodiments, known momentary radial force exerted by scaffold can include characterizing self-expanding stent and map force to expansion diameter, relatively stiff foreshortening scaffold is paired with a spring in series (either within handle or within scaffold). Force versus expansion can characterized and projected onto a high/low indicator in handle. Spring compression/force could also be measured by a sensor and transmitted electronically to handle. In some embodiments, the devices disclosed herein can include one or more variable capacitors, piezoelectric pressure sensors, linear positional transducers coupled to spring. In some embodiments, a rigid expansion mechanism (scissor-lift scaffold) is paired with solid-state piezoelectric force transducer (or PZT force transducer). In some embodiments, foreshortening is converted to known expansion and force is measured electronically.

In some embodiments, foreshortening stent scaffold with mechanical force measurement can include foreshortening stent shaped scaffold that is laser cut from nitinol tube. Integrated spring can allow for additional compressibility and for increased force resolution.

In some embodiments, the techniques described herein relate to a catheter device for determining a force required to radially expand a blood vessel of a subject to an indicated diameter while maintaining blood flow, the device including: a body portion, including: a handle body; an actuator configured to create a linear motion; a plurality of sensors located within the handle body, the sensors including force sensing and displacement sensing; a scaffold configured to expand and contract in a transverse direction, the scaffold including a plurality of struts, the scaffold configured to be variable in expansion and contraction movement between a collapsed first position and an expanded second position; a shaft assembly positioned in the catheter device such that a proximal end of the shaft assembly is coupled with the proximal end of the body portion, the shaft assembly connecting the body portion to the scaffold and configured to translate force between the body portion and the scaffold, the shaft assembly including: an outer shaft; an inner shaft within the outer shaft; and a central lumen defined by a channel in the inner shaft and configured to receive a guidewire; wherein, the transverse direction is transverse relative to a longitudinal axis of the shaft assembly, the force sensing is configured to contact a shaft adapter, and the plurality of sensors are configured to measure force and displacement applied by the blood vessel to the scaffold and translated though the shaft.

In some embodiments, the techniques described herein relate to a catheter device, wherein in the collapsed first position, the scaffold includes a first length and a first radial diameter, and in the expanded second position, the scaffold includes a second length and a second radial diameter, the second length shorter than the first length, the second radial diameter larger than the first radial diameter.

In some embodiments, the techniques described herein relate to a catheter device, including a shuttle coupled to the actuator, the shuttle configured to advance or retract responsive to activating the actuator.

In some embodiments, the techniques described herein relate to a catheter device, wherein the shuttle includes a slot, the displacement sensor includes a post, the post of the displacement sensor configured to be positioned in the slot of the shuttle.

In some embodiments, the techniques described herein relate to a catheter device, wherein the force sensing is positioned between the adapter of the outer shaft and a portion of the shuttle.

In some embodiments, the techniques described herein relate to a catheter device, wherein the force sensing includes a piezoresistive sensor.

In some embodiments, the techniques described herein relate to a catheter device, wherein the displacement sensor is a slide potentiometer.

In some embodiments, the techniques described herein relate to a catheter device, wherein the catheter device is configured such that the scaffold or the shaft assembly does not include a sensor.

In some embodiments, the techniques described herein relate to a catheter device, further including a digital display configured to provide one or more of: an indication of a diameter of the scaffold, an axial displacement of the shaft assembly, a constrictive wall force of the blood vessel, and/or an axial force on the scaffold.

In some embodiments, the techniques described herein relate to a catheter device, wherein each of the plurality of struts includes branching arms to promote preferential radial expansion of the scaffold while maintaining an apex portion of the scaffold with a profile that is approximately: flat, concave, or convex.

In some embodiments, the techniques described herein relate to a catheter device, wherein the actuator is one of a knob, a slide, or a motor.

In some embodiments, the techniques described herein relate to a catheter device, further including a second plurality of sensors positioned at a distal end of the shaft assembly.

In some embodiments, the techniques described herein relate to a catheter device for determining a force required to radially expand a blood vessel of a subject to an indicated diameter while maintaining blood flow, the device including: a body portion, including: a handle body; an actuator configured to create a linear motion; a scaffold configured to expand and contract in a transverse direction, the scaffold including a plurality of struts, the scaffold configured to be variable in expansion and contraction movement between a collapsed first position and an expanded second position; a shaft assembly positioned in the catheter device such that a proximal end of the shaft assembly is coupled with the proximal end of the body portion, the shaft assembly connecting the body portion to the scaffold and configured to translate force between the body portion and the scaffold, the shaft assembly including: an outer shaft; an inner shaft within the outer shaft; and a central lumen defined by a channel in the inner shaft and configured to receive a guidewire; a plurality of sensors, including: a force sensor positioned in one of: the shaft assembly; partially in the shaft assembly and body portion; or partially in the scaffold, shaft, and body portion; a displacement sensor positioned in one of: the shaft assembly; partially in the shaft assembly and body portion; or partially in the scaffold, shaft assembly, and body portion; wherein, the transverse direction is transverse relative to a longitudinal axis of the shaft assembly, and plurality of sensors are configured to measure force and displacement applied by the blood vessel to the scaffold and translated though the shaft.

In some embodiments, the techniques described herein relate to a catheter device, wherein the plurality of struts are coupled to the outer shaft at a proximal end of the scaffold and to the inner shaft at a distal end of the scaffold.

In some embodiments, the techniques described herein relate to a catheter device, wherein the plurality of struts are coupled to the inner shaft and the outer shaft with a mechanically restraining component.

In some embodiments, the techniques described herein relate to a catheter device, wherein the mechanically restraining component includes a plurality of receiving features configured to mate with the plurality of struts.

In some embodiments, the techniques described herein relate to a catheter device, wherein the force sensor and the displacement sensor are communicatively coupled to a processor.

In some embodiments, the techniques described herein relate to a catheter device, wherein the processor is configured receiving an analog signal through the shaft assembly, convert the analog signal to a digital signal, and calibrate the digital signal to determine a force required to radially expand the vessel of a subject.

In some embodiments, the techniques described herein relate to a catheter device, wherein each of the plurality of struts includes branching arms to promote preferential radial expansion of the scaffold while maintaining an apex portion of the scaffold with a profile that is approximately: flat, concave, or convex.

In some embodiments, the techniques described herein relate to a catheter device, further including a digital display configured to provide one or more of: an indication of the diameter of the scaffold, the axial displacement of the shaft assembly, the constrictive wall force of the blood vessel, and/or the axial force on the scaffold.

In some embodiments, the techniques described herein relate to a catheter device, wherein the catheter device is configured such that the scaffold does not include a force sensor or a displacement sensor.

In some embodiments, the techniques described herein relate to the displacement sensor does not include a spring.

### BRIEF DESCRIPTION OF THE DRAWINGS

These and other features, aspects, and advantages of the present disclosure are described with reference to the drawings of certain embodiments, which are intended to illustrate certain embodiments and not to limit the invention, in which like reference numerals are used for like features, and in which:

FIG. 1 illustrates various components of a catheter device according to some embodiments of the present disclosure.

FIGS. 2A through 2B illustrate some components of the catheter device according to some embodiments of the present disclosure.

FIGS. 3A through 3B illustrate a top view of some components of the catheter device according to some embodiments of the present disclosure.

FIGS. 4A through 4B illustrate a side view of some components of the catheter device according to some embodiments of the present disclosure.

FIGS. 5A through 5D illustrate some components of a scaffold assembly according to some embodiments of the present disclosure.

FIGS. 6A through 6C illustrate a side view of a scaffold at various stages of expansion according to some embodiments of the present disclosure.

FIG. 7 illustrates a side cross sectional view of some components of the catheter device, including the scaffold and a shaft, according to some embodiments of the present disclosure.

FIGS. 8A through 8C illustrate a display of the catheter device according to some embodiments of the present disclosure.

FIG. 9 illustrates a signal flow chart according to some embodiments of the present disclosure.

FIGS. 10A-10B illustrate various performance measurements of some embodiments of the present disclosure.

FIG. 11 illustrates a catheter device according to some embodiments of the present disclosure.

FIG. 12 illustrates a catheter device with guidewire lumen according to some embodiments of the present disclosure.

FIG. 13 illustrates a catheter device including a valve according to some embodiments of the present disclosure.

FIG. 14 illustrates a catheter device with guidewire lumen according to some embodiments of the present disclosure.

FIG. 15 illustrates a force indicator on a handle of a catheter device according to some embodiments of the present disclosure.

FIGS. 16-18 illustrate examples of a stent scaffold according to some embodiments of the present disclosure.

### DETAILED DESCRIPTION

The various devices, systems and methods disclosed herein are suited to use as a diagnostic catheter system. Advantageously, various embodiments of such devices, systems and/or methods can permit for expansion of vascular lumen and measurement of radial forces exerted by the vessel wall during such expansion. The embodiments disclosed herein offer one or more advantages and benefits over existing technologies, such as, for example and without limitation, facilitating non-occlusive expansion of vascular lumen, calibrating and measuring various parameters (e.g., non-linear force sensitivity, non-linear compensation for scaffold diameter, etc.), providing components (e.g., scaffold, shaft, etc.) with characteristics that can be tuned for application-specific needs, providing efficient sensor configuration (e.g., sensor configuration is not sensitive to contact a tip of the device, sensor is positioned in the handle body, etc.), facilitation expansion of the scaffold in a controlled and direct manner, and/or the like, and other advantages/benefits.

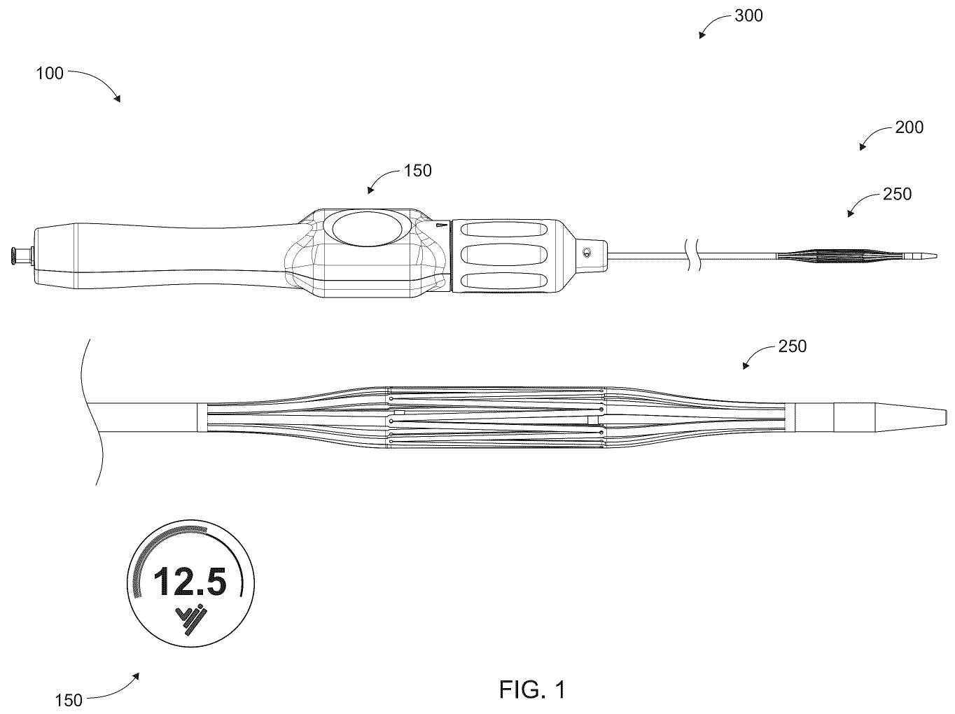

FIG. 1 illustrates various components of a catheter device according to some embodiments of the present disclosure. The catheter device 300 can be used in a variety of medical applications. For example, the catheter device 300 can assist in venous stenting procedures. The catheter device 300 can include a body portion 100 and catheter portion 200. In some embodiments, the catheter portion 200 can include a scaffold 250 with mechanical force measurement means. The scaffold 250 can be communicatively coupled to a controlled deployment mechanism in the body portion 100, enabling the scaffold 250 to expand and provide force feedback measurement to the user. The scaffold 250 can be configured such that it does not include a force sensor and/or a displacement sensor.

As illustrated in FIG. 1, in some embodiments, the body portion 100 can include a display 150 configured to provide feedback (e.g., deployed diameter, compressive force, etc.) to a user operating the catheter device 300.

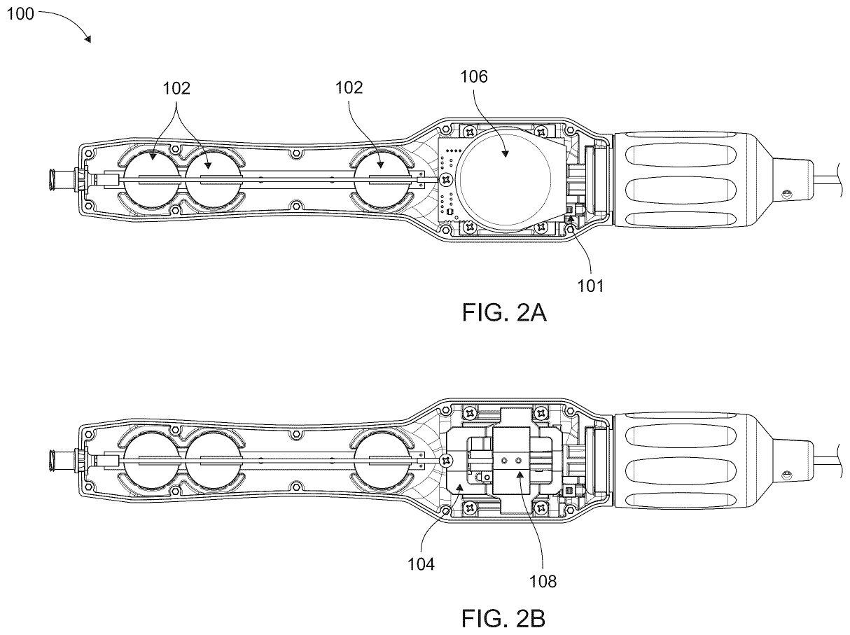

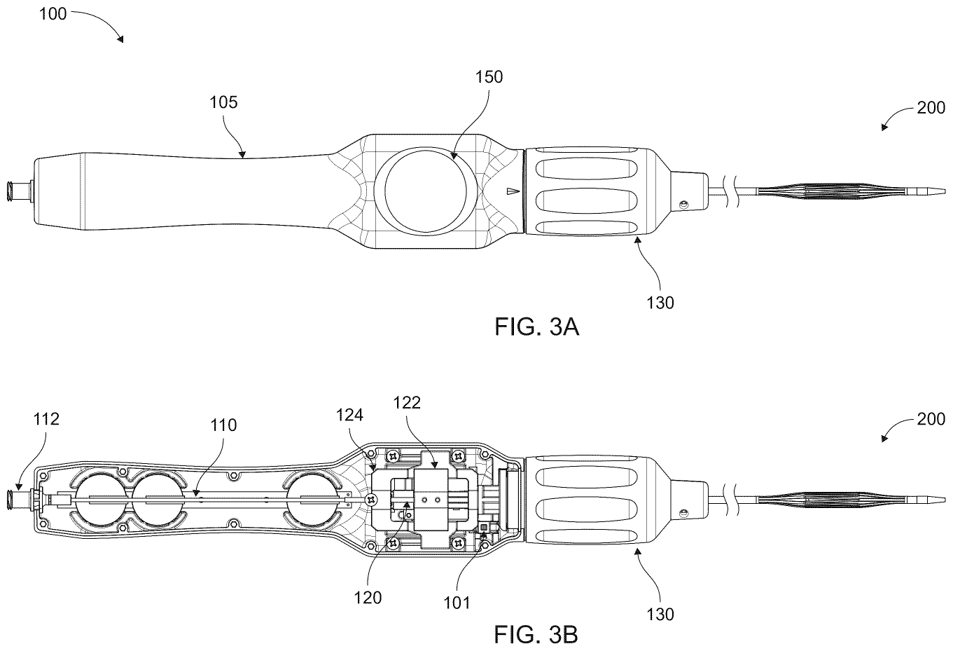

FIGS. 2A through 2B illustrate some components of the catheter device 300, including components configured to be positioned inside the body portion 100. For illustration purposes, a portion of the handle shell (see, e.g., handle shell 105 in FIG. 3A) is not shown. The body portion 100 can include an on-switch 101, and one or more batteries 102. The one or more batteries 102 (e.g., one battery, two batteries, three batteries, six batteries, etc.) can provide the necessary power for a self-contained operation. For example, the one or more batteries 102 can allow the user to utilize the catheter device 300 without the need to be constrained by a power cable, thereby facilitating a more convenient and efficient operation.

FIGS. 2A through 2B also illustrate a shaft displacement shuttle 104, a processor board 106, and an outer shaft coupler 108. The processor board 106 can translate the feedback to an indicator (e.g., a colored indicator) in the display 150, enabling visualization of the force(s) applied and/or other measurements. As will be discussed in more detail elsewhere in the present disclosure, these components can cooperate with one another (and with other components not illustrated in FIGS. 2A through 2B) to facilitate the operation of the catheter device 300.

FIGS. 3A through 3B illustrates a top view of some components of the catheter device 300. More specifically, FIG. 3A illustrates a handle shell 105, a display 150, and an actuator. Although the actuator is illustrated as a knob 130, other suitable actuators (e.g., motor, slide, gear track, linear gear train, etc.) can also be used to accommodate a user's need. The handle shell 105 can be sized for comfort depending on the user's preference. In one example, the display 150 includes a Liquid Crystal Display (LCD) screen. The display 150 can be located between the knob 130 and the handle shell 105 to facilitate an intuitive two-handed operation.

In some embodiments, the handle shell 105 can be split into two mating clamshells (e.g., a top half and bottom half) for assembly and/or case of maintenance. FIG. 3B illustrates the catheter device 300, with the top half of the handle shell 105 removed for illustrative purposes. As illustrated in FIG. 3B, the body portion 100 can include a luer interface 112, an inner shaft 110, an outer shaft 120, an adapter component, or an outer shaft adapter 122, and a shuttle 124. The knob 130 is positioned at a distal end of the body portion 100. The knob 130 can provide an actuation mechanism. In one example, the knob 130 can be over molded Santoprene™ (or other similar materials). In some embodiments, turning the knob 130 (e.g., in the clockwise direction or in the anticlockwise direction, or otherwise) can drive the outer shaft 120 forward.

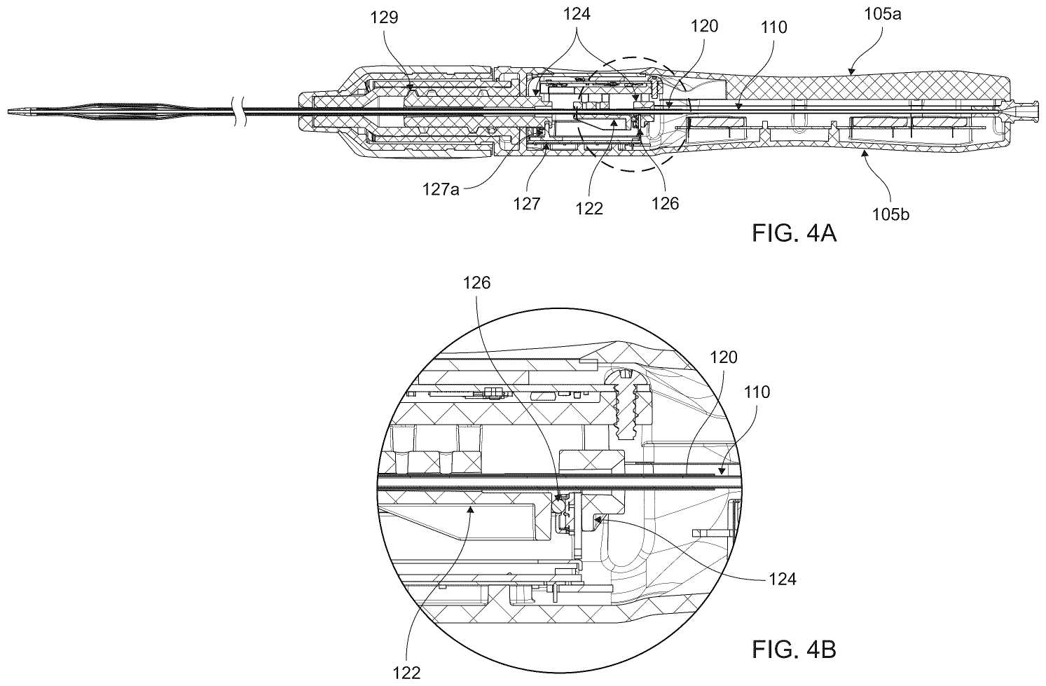

As illustrated in FIG. 3B, the catheter device 300 can include the inner shaft 110, luer interface 112, outer shaft 120, the outer shaft adapter 122, the shuttle 124, the knob 130, and the scaffold 250. In some embodiments, the inner shaft 110 is coupled to the luer interface 112, and the outer shaft 120 is coupled to the adapter component 122. The catheter device 300 can be activated by engaging the knob 130. For example, the activation of the catheter device 300 can be initiated by a tactilely indexed rotation of the knob 130, triggering the on-switch 101, which can trigger an electronic startup sequence. In some embodiments, the rotation may be a 45-degree rotation. In some embodiments, the knob 130 can be configured such that when the knob 130 is rotated (e.g., in a clockwise or anticlockwise orientation), a lead screw positioned within the knob 130 advances the shuttle 124. In some embodiments, the shuttle 124 can include one or more force sensors (e.g., a piezoelectric strain gauge or piezoresistive force sensors 126 illustrated in FIG. 4B) communicatively coupled to a processor. Accordingly, the one or more force sensors can push against the outer shaft adapter 122, registering force as the shaft advances and the scaffold 250 expands. Once activated, the catheter device 300 remains energized until the one or more battery 102 is depleted or a self-destruct lockout function is triggered.

FIGS. 4A through 4B illustrate a side view of some components of the catheter device 300, including top handle shell 105a, a bottom handle shell 105b, an inner shaft 110, an outer shaft 120, an outer shaft adapter 122, a shuttle 124, one or more force sensors 126, a displacement sensor or linear translation sensor 127 (e.g., slide potentiometer, variable resistor, etc.) having a post 127a, and a lead screw positioned within the knob 130, according to some embodiments of the present disclosure. The displacement sensor can be configured to measure axial forces transmitted by the scaffold through the inner shaft 110 and/or the outer shaft 120. In some embodiments, a linear displacement sensor (e.g., the variable resistor disclosed herein) measures the relative movement of the outer shaft with respect to the fixed inner shaft. Although, in some embodiments, the inner shaft can be fixed, the devices of the present disclosure can also be configured such that the outer shaft is fixed. For example, the inner shaft can pull the scaffold in, allowing for expansion of the scaffold, while the outer shaft is fixed. As discussed above, the catheter device 300 can be configured to provide feedback measurement on one or more parameters (e.g., force, diameter, etc.) as the scaffold 250 is deployed in the body of a subject. For example, the catheter device 300 can measure a force transmitted by the outer shaft 120 and provide feedback corresponding to the forces exerted at a blood vessel wall. More specifically, when the knob 130 is rotated, the knob helix 129 advances (e.g., moves) the shuttle 124 distally in a controlled manner. The piezoresistive force sensor 126 is pressed against the outer shaft adapter 122, thereby measuring the force transmitted by the outer shaft 120. In one example, the catheter device 300 can measure the diameter of the stent scaffold as the stent scaffold deploys (e.g., expands) in the vascular lumen. More specifically, in some embodiments, the post 127a of the slide potentiometer 127 is coupled to and/or is positioned in a slot in the shuttle 124. As the shuttle 124 is advanced or retracted by the knob helix 129, the post 127a actuates the slide potentiometer 127 to measure the axial shaft displacement between the outer shaft 120 and inner shaft 110, thereby measuring the diameter of the stent scaffold.

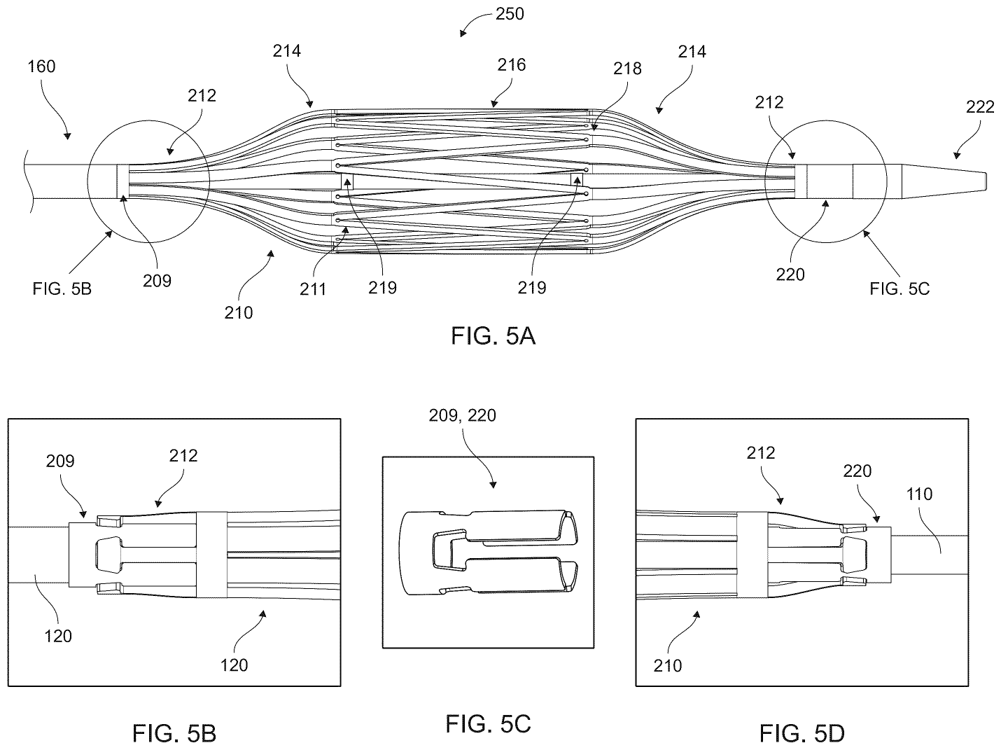

FIG. 5A illustrates a side view of the scaffold 250 of the catheter device 300. The scaffold 250 can be manufactured from one or more materials (nitinol, and/or other biocompatible materials) in different sizes (e.g., having a length of about 20 millimeters, 30 millimeters, 40 millimeters, etc.). The scaffold 250 can include a plurality of splines 210. For example, the scaffold 250 can include six, seven, eight, nine, twelve, fourteen splines or a different number of splines. In one example, the scaffold 250 includes eight splines. The splines 210 can be axially oriented and/or extend longitudinally between the outer shaft 120 and the distal tip 222. Each of the plurality of splines 210 can be coupled to two expanding or branching arms 211. In some embodiments, the expanding or branching arms 211 can have a sinusoidal configuration. The geometry of the splines 210 and expanding or branching arms 211 can be configured such that, in some embodiments, the transitions at both the shaft connection 212 and the spline transition to apex portion 214 are tangent to the axial profile. Advantageously, the geometry of splines 210 and expanding or branching arms 211 improve the transformation of radial vessel wall forces into axial loading while maintaining balanced radial strength and ensuring sufficient vessel wall coverage to prevent prolapse.

Still referring to FIG. 5A, the scaffold 250 can have an apex portion 216. The apex portion 216 can be horizontal or substantially horizontal. In use, the apex portion 216 can be the upper most portion of the scaffold 250. In some embodiments, the apex portion 216 of the scaffold 250 can have a length of about 20 mm at full expansion. In some embodiments, the apex portion 216 can be configured such that it is substantially flat (e.g., horizontal, or substantially parallel with the inner shaft 110). Advantageously, this allows the scaffold 250 to establish a known and consistent contact length with the vessel wall of the subject, thereby improving the feedback measurements.

Still referring to FIG. 5A, the scaffold 250 can include a spline transition to apex portion 214. The spline transition to apex portion 214 can facilitate the transition of the spline 210 at the apex portion 216. In some embodiments, the spline transition to apex portion 214 is tangent to the axial profile. Advantageously, the tangent orientation of the spline transition to apex portion 214 facilitates an atraumatic geometry, allowing the catheter device 300 to operate while reducing any unintended impact to the surrounding biological tissues.

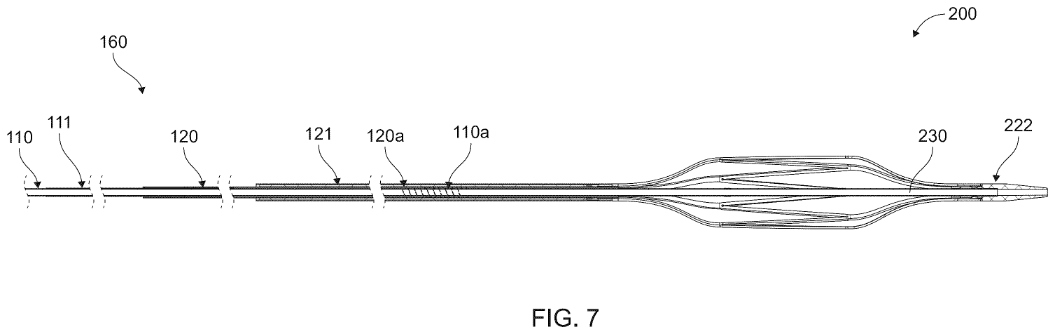

FIGS. 5B and 5D illustrate some components of the catheter device 300 transitioning from the scaffold 250 to a shaft assembly 160 (e.g., to an outer shaft 120 or an inner shaft 110). For illustration purposes, some components of the shaft assembly 160 (e.g., see jacket 121 illustrated in FIG. 7) are not shown. In some embodiments, the catheter device 300 can include one or more mechanically restraining component (also referred to as scaffold cuffs), such as a proximal scaffold cuff 209 and/or a distal scaffold cuff 220 illustrated in FIG. 5C. In one example, the scaffold cuff(s) can be manufactured as continuous tubular portions to provide enhanced strength at the shaft connection points (e.g., at the connection to the outer shaft 120 and the inner shaft 110), which can experience the highest mechanical loads when the catheter device 300 is in use. The scaffold cuffs can include one or more openings configured to mate with the cuff transition 212 of the spline 210, thereby securing the spline 210 to the shaft assembly 160. As illustrated in FIG. 5B, the proximal scaffold cuff 209 is secured to the outer shaft 120, thereby securing a proximal end of the scaffold 250. FIG. 5D illustrates transition of the spline 210 to the distal scaffold cuff 220 through the cuff transition 212. The distal scaffold cuff 220 can secure a distal end of the scaffold 250 to the inner shaft 110. The cuff transition 212 can be configured to efficiently transfer axial forces from the vessel wall of a subject into the shaft assembly through the proximal scaffold cuff 209 and/or the distal scaffold cuff 220.

As discussed above, in one example, each of the plurality of splines 210 can be coupled to two expanding or branching arms 211 at an expanding joint. In some embodiments, one or more openings 218 (e.g., a hole feature) can be incorporated at the expanding joints of the scaffold 250. Advantageously, the one or more openings 218 can promote preferential radial expansion of the scaffold 250 while maintaining an approximately flat profile at the apex portion 216.

With continued reference to FIG. 5A, the shaft assembly 160 can include a distal tip 222. In some embodiments, the distal tip 222 of the shaft assembly 160 is tapered (e.g., includes a reduced cross section). Advantageously, this provides an atraumatic transition from the guidewire to the outer diameter of the inner shaft 110. One or more radiopaque markers 219 can be positioned on the inner shaft 110 to indicate the locations of the scaffold 250 corners at a point of expansion (e.g., at the point of maximum expansion).

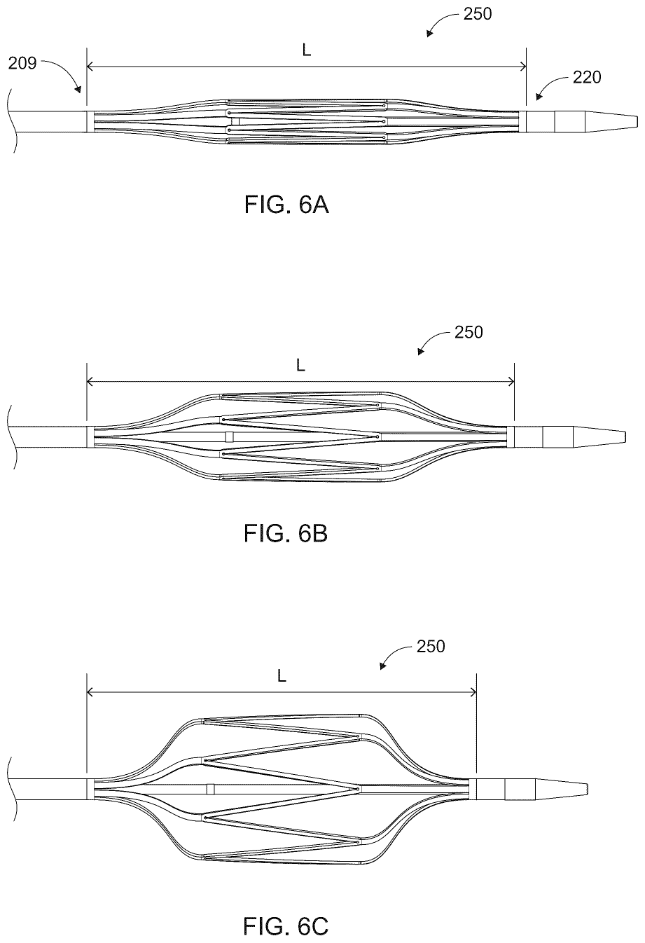

FIGS. 6A-6C illustrate a side view of the scaffold 250 at various stages of expansion according to some embodiments of the present disclosure. More specifically, FIG. 6A illustrates the scaffold 250 at an unconstrained stage; FIG. 6B illustrates the scaffold 250 at an intermediately expanded stage; and FIG. 6C illustrates the scaffold 250 at an expanded stage. As the catheter device 300 operates and the outer shaft 120 starts to move relative to the inner shaft 110, a length L (corresponding to the length between about proximal scaffold cuff 209 and the distal scaffold cuff 220) reduces while the outer diameter of the scaffold increases. In one example, the scaffold 250 can have a length L of about 2.384 cm and an outer diameter of about 6 mm at an unconstrained stage; a length L of about 2.321 cm and an outer diameter of about 12 mm at an intermediately expanded stage; and a length L of about 2.2.120 cm and an outer diameter of about 20 mm at an expanded stage. Accordingly, the scaffold 250 can have an unconstrained length smaller or larger than about 2.384 cm (e.g., 2.321 cm, 2.5 cm, 2.7 cm, 3.1 cm), an intermediately expanded length L smaller or larger than about 2.321 cm (e.g., about 2.234 cm, 2.384 cm, 2.521 cm, 3.123 cm, etc.), or an expanded length L smaller or larger than about 2.120 cm (e.g., about 2 cm, 2.123 cm, 2.321 cm, 3.231 cm, etc.). Similarly, the scaffold 250 can have an unconstrained outer diameter smaller or larger than about 6 mm (e.g., about 4 mm, 4.5 mm, 6.5 mm, 7 mm, etc.), an intermediately expanded outer diameter smaller or larger than about 12 mm (e.g., about 11 mm, 12.5 mm, 13.5 mm, etc.), or an expanded outer diameter smaller or larger than about 20 mm (e.g., about 19 mm, 19.5 mm, 21 mm, 22 mm, etc.). Advantageously, this allows the scaffold 250 to accommodate anatomical variations of a patient and/or accommodate different patients with varying luminal diameters. As the scaffold 250 expands, the radial compressive luminal force is transformed into an axial force between the inner and outer shaft.

As illustrated in FIGS. 6A through 6C, the scaffold 250 can, in some embodiments, include a laser cut pattern or configuration. Advantageously, the laser cut pattern facilitates diametral expansion of the scaffold 250 when the axial length of the scaffold is reduced. In addition, such laser cut pattern can reduce the force required to deploy the catheter device 300 and allow for more sensitivity to the compressive luminal forces.

As illustrated in FIGS. 6A-6C and discussed above, in one example, the scaffold 250 can include eight axial arms or splines 210 that transition into sixteen linked expanding or branching arms 211, arranged in a sinusoidal orientation. Advantageously, such arrangement of the splines and expanding arms can keep radial symmetry to distribute the radial forces onto the shaft assembly 160. Furthermore, such arrangement of splines 210 and expanding or branching arms 211 can allow for sufficient wall coverage to prevent vein prolapse through the scaffold during expansion. Each spline 210 can have a wall thickness of about .010 inch (corresponding to about 0.254 mm) or about 0.025 inch (corresponding to about 0.635 mm) or other values smaller than about .010 inch (about 0.254 mm) or larger than about. 025 inch (about 0.635 mm) or any other value therebetween. Each spline 210 can be configured to include sufficient strength to expand compressed veins. Each spline 210 can be configured to allow for blood flow therethrough, thereby providing a non-occluding catheter device.

FIG. 7 illustrates various components of the shaft assembly 160, including an inner shaft 110, an outer shaft 120, and an inner shaft sleeve 111 between the inner shaft 110 and the outer shaft 120. The inner shaft sleeve 111 can include one or more polymers (e.g., polyimide). In one example, the inner shaft sleeve is a polyimide sleeve. Advantageously, the polyimide sleeve can help keep the inner shaft 110 and the outer shaft 120 aligned and/or protect electronics from shorting to the shaft assembly 160. The inner shaft 110 can be coaxial with the outer shaft 120 and/or the inner shaft sleeve 111.

Still referring to various components of the shaft assembly 160, the shaft assembly 160 can include one or more cut features 110a (e.g., groove, notch, cut, carve, etc.), along the length of the inner shaft 110. In one example, the one or more cut features 110a can include a laser cut pattern. In one example, a distal portion of the inner shaft 110 includes one or more cut features 110a while the proximal portion of the inner shaft 110 includes a reduced number of cuts (e.g., no cut). The shaft assembly 160 can include one or more cut features 120a (e.g., groove, notch, cut, carving, etc.) along the length of the outer shaft 120. In one example, the one or more cut features 120a can include a laser cut pattern. In one example, a distal portion of the outer shaft 120 includes one or more cut features 120a while the proximal portion of the outer shaft 120 includes a reduced number of cuts (e.g., no cut). Advantageously, the one or more cut features 110a and/or the one or more cut features 120a can help balance flexibility for deliverability, and axial stiffness for force transfer.

Also illustrated in FIG. 7 are a shaft jacket 121, an inner guidewire lumen 230, and a distal tip 222. The shaft jacket 121 can provide an atraumatic surface finish to reduce the adverse impact of the catheter portion 200 during a medical procedure. The inner guidewire lumen 230 can house a guidewire. The guidewire (not shown) can be used to guide the catheter portion 200 to the target lesion. The distal tip 222 can be an atraumatic tip. In some embodiments, the distal tip 222 is a polymer atraumatic tip.

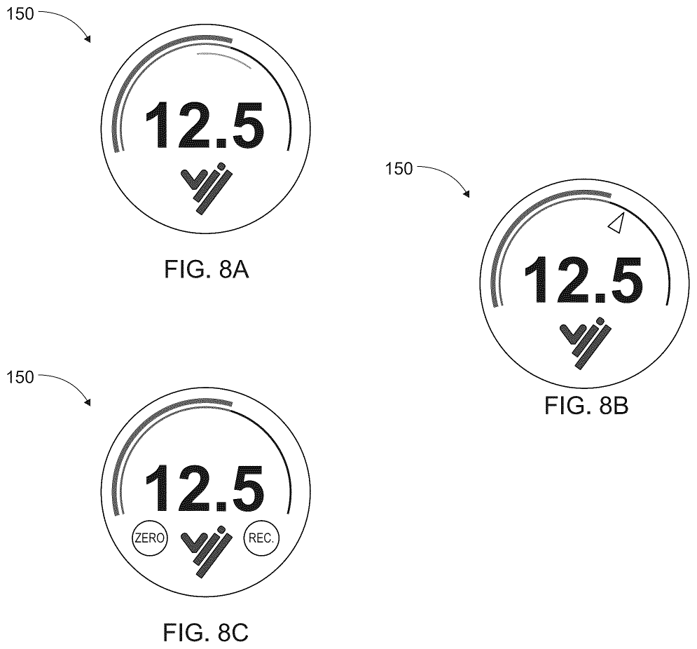

FIGS. 8A through 8C illustrate various embodiments of a display 150 of the catheter device 300 according to some aspects of the present disclosure. In some embodiments, the catheter device 300 uses a high pixel density LCD screen to display various measurements and/or other relevant information to the user. The display 150 can include a user interface configured to provide one or more information to the user and/or receive input from the user. The display 150 can be configured to allow for flexibility of communication. For example, the display 150 can include features to allow the user to easily switch between different languages, provide non-linear color bar scaling, include re-zero indicators, include peak hold indicators, etc. In some embodiments, the display 150 can show force range over time intervals (e.g., about 1s to about 10s) to display for dynamic force changes from respiration, movement, etc. as illustrated in FIG. 8A. In some embodiments, the display 150 can be movable (e.g., can rotate) to provide optimal visualization during a medical operation. The display 150 can provide a guided field calibration of scaffold deployment forces. The display 150 can include a peak hold indicator (see, e.g., FIG. 8B). Referring to FIG. 8C, the display 150 can include a touch screen interface for user inputs (e.g., zero/tare button, color scale adjustments, etc.), and/or include features to record time-stamped force data for patient records.

In the examples of FIGS. 8A through 8C, the display 150 illustrates a scaffold diameter of 12.5 mm, and a force indicator bar is positioned above the scaffold diameter. The digital signal processing of the present disclosure can enable the force indicator bar to have one or more color (e.g., yellow, green, red, etc.), allowing the user to quickly assess the load that the scaffold is subject to. For example, a green indicator can convey to the user that the scaffold is not subject to an adequate pressure that is needed to secure the stent in the desired location. Advantageously, this feature reduces the risks associated with migration of stents from a desired due to insufficient force, or collapse of the stent as a result of excessive forces on the stent.

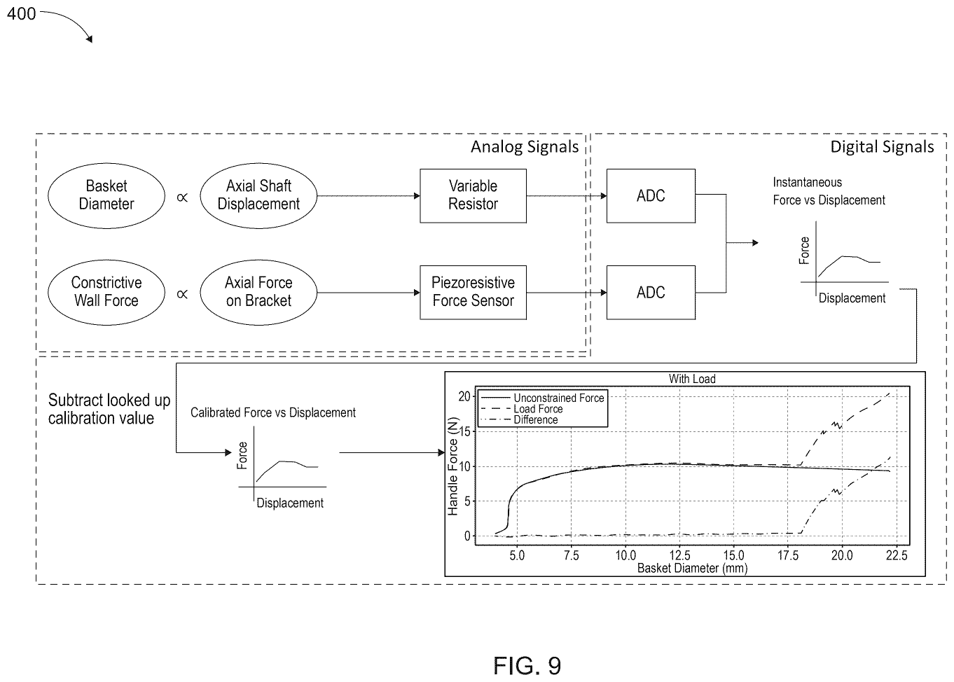

FIG. 9 is an example flow chart illustrating signal processing features of the present disclosure according to some embodiments. As discussed above and illustrated in FIG. 9, the devices of the present disclosure can use a combination of analog signals, digital signals, and/or calibration methods to more accurately measure forces applied to the stent scaffold during an operation. For example, the processor disclosed herein can be configured to obtain various analog signals (e.g., analog signals indicative of scaffold diameter, axial shaft displacement, constrictive wall force, and/or axial force on scaffold) utilizing one or more variable resistor and/or a piezoresistive force sensor. In some embodiments, an analog signal detected by a variable resistor may be indicative of a scaffold diameter, which may be directly proportional to the axial shaft displacement. In some embodiments, an analog signal detected by a force sensor (e.g., a piczoresistive force sensor) may be indicative of a constrictive wall force, which may be directly proportional to the axial force on scaffold. An Analog-to-Digital Converter (ADC) can be used to convert the analog signals from the variable resistor and/or the force sensor to digital signals, thus allowing said signals to be processed, stored, and/or manipulated by the processor. The processor disclosed herein can be configured to utilize digital signals to generate a relationship between an instantaneous force on the scaffold and a displacement of the axial shaft. In some embodiments, the relationship may include an instantaneous force vs. displacement curve. In some embodiments, a memory device of the processor may store calibration values. The processor may perform calibration on the detected signals (e.g., by subtracting a calibration value from a look up table or applying a calibration equation) to obtain a calibrated relationship of force versus displacement (e.g., stored as a calibrated force vs. displacement curve) that reflects the calibrated measurement of forces applied to the scaffold as the scaffold diameter increases.

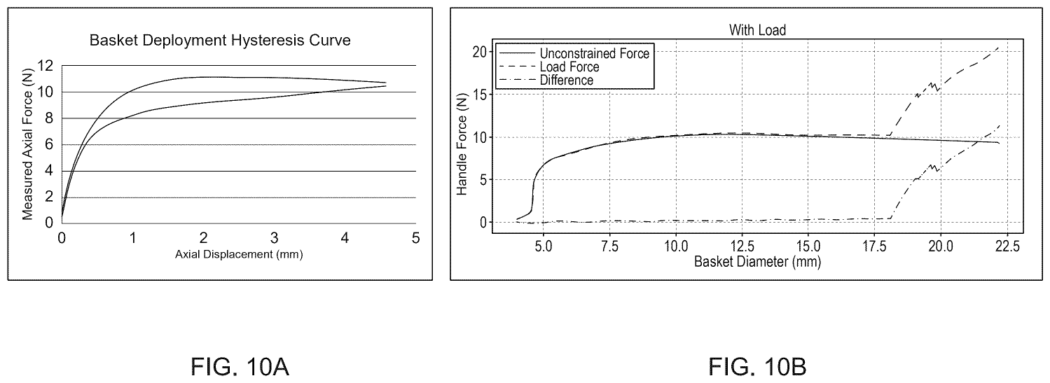

FIGS. 10A through 10B illustrate various performance measurements of some embodiments of the present disclosure, including one or more calibration curves stored in the memory of a processor. As illustrated in FIG. 10A, the force and axial displacement measurements can be processed to generate a deployment curve (displacement vs. force). In some embodiments, such a deployment curve can be stored as a lookup table or a calibration curve to compensate for scaffold-specific non-linearities and hysteresis. In some embodiments, in use, the processor disclosed herein dynamically samples displacement and force data, applies calibration compensation, and outputs vessel compressive force and compensated diameter measurements on the integrated LCD display. In some embodiments, the catheter device 300 can include one or more strain gauge sensors positioned proximal to the scaffold and distal to the flexible shaft zone, and/or capacitive strain sensors, and/or fiber optic strain sensors (e.g., fiber Bragg grating, refractometry-based systems, etc.). FIG. 10B is an example graph illustrating the increase in the handle force as the scaffold diameter increases according to some embodiments. More particularly, FIG. 10B illustrates the compensation for non-linear scaffold deployment force. For example, the initial opening of a scaffold requires a baseline force. In some embodiments, the processor of the present disclosure can be configured to subtract such baseline force from the force measured as the scaffold contacts the vein wall in order to accurately represent the additional force that is measured from contact with the vein wall. FIG. 10B illustrates a plot of the following parameters: Unconstrained Force, Load Force, and Difference. The Difference (which is the true wall force on the device) is the difference between the Load Force and the Unconstrained Force.

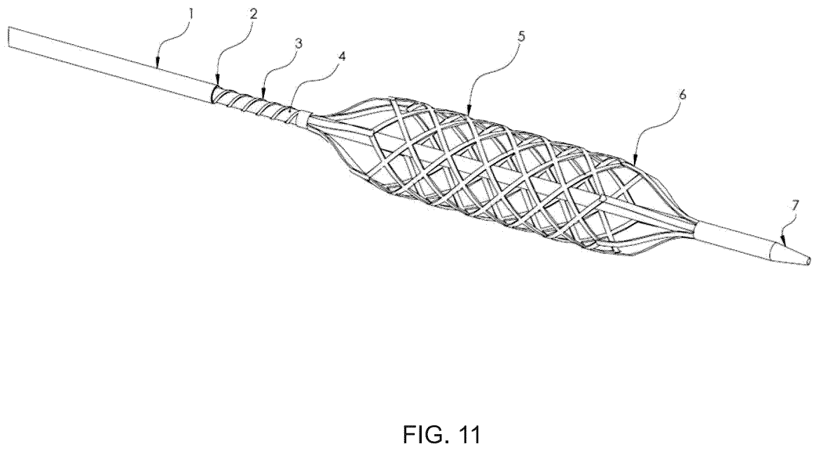

FIG. 11 illustrates a catheter device with an outer shaft 1. The outer shaft 1 can be a braided, lined 8.5F shaft, proximally connected to handle mechanism, and distally reflowed over proximal end of scaffold. The catheter device of FIG. 11 can have an outer shaft to scaffold connection 2, which can be shaft reflowed over proximal end of stent mechanism to mechanically link scaffold to shaft. The catheter device of FIG. 11 can include a scaffold spring 3. The scaffold spring 3 can be laser cut compression spring-strut thickness tuned such that the spring is slightly less compressible than the scaffold-scaffold expands first, and then force can be applied to spring once sufficient wall force is experienced. In some embodiments, length can be 4 cm at full expansion. The catheter device of FIG. 11 can include an inner shaft 4. The inner shaft 4 can be an inner braided shaft that stabilizes scaffold and spring-acts as inner piston for spring control. Scaffold can be distally connected to inner shaft, which does not shorten. Inner shaft can contain central lumen for up to 0.035″ guidewire passage. See additional detail in cross section. The catheter device of FIG. 11 can include an expansion scaffold 5. The expansion scaffold 5 can be sufficiently rigid such that it does not buckle under higher-than-normal wall forces. The catheter device of FIG. 11 can include connector struts 6. The connector struts 6 can link the scaffold to the shaft bodies. The connector struts 6 can include a plurality of struts. These struts can be the main way that the shaft displacement is translated to radial expansion. The connector struts 6 can be sufficiently strong so as not to buckle under higher-than-normal wall forces. The catheter device of FIG. 11 may include two layers of struts if required. The catheter device of FIG. 11 can include a shaft distal tip 7. The shaft distal tip 7 can be tapered for atraumatic access. In some embodiments, the shaft distal tip can contain central lumen for acceptance of up to 0.035″ guidewire.

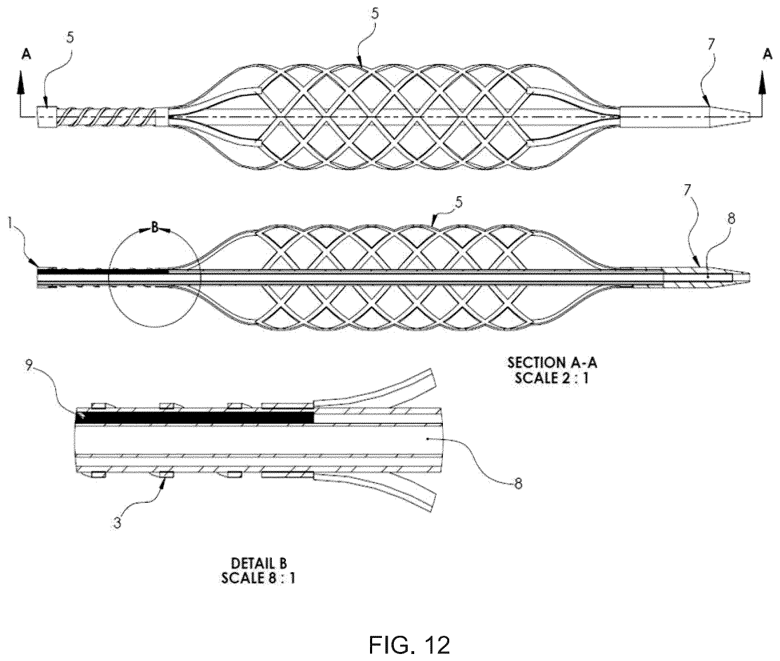

FIG. 12 illustrates a catheter device with guidewire lumen 8. In some embodiments, the lumen can be sized 0.038″ ID to accept up to 0.035″ wire. The lumen can be made of Nylon. FIG. 12 also illustrates a spring compression translation wire 9. In some embodiments, small diameter spring tempered wire is attached to the distal end of the scaffold compression spring 3 and is routed through reinforced lumen in shaft to an indicator sight in the handle 13. The spring compression translation wire 9 can allow for visualization of state of spring compression.

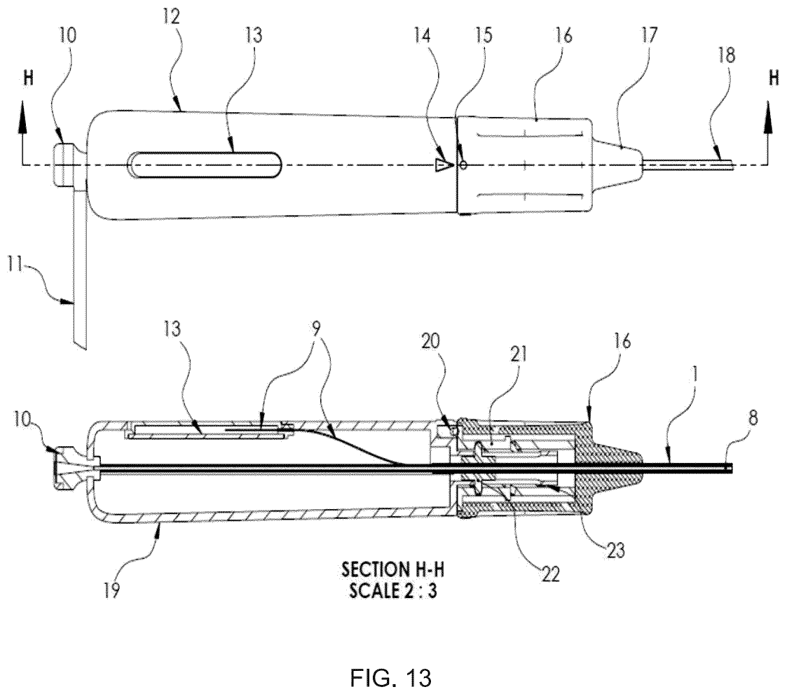

FIG. 13 illustrates a catheter device including hemostasis valve 10. The hemostasis valve 10 can be for guidewire access. The catheter device of FIG. 13 can have side port tube 11, which can be a side port tube to 3-way stopcock for flushing catheter. The catheter device of FIG. 13 can include a handle A-half 12. The body of handle can be sized for comfort when using the product. A force indicator sight glass 13 can have a transparent body with green/yellow/red color indicators below it. The spring compression translation wire 9 can be routed into sight and can be free to move with the compression/extension of the attached spring. A handle position indicator 14 can indicate current scaffold expansion. A rotary knob zero indicator 15 can indicate position at which scaffold expansion is zero. Knob can be numbered, for example, “12”, “14”, “16”, “18”, to indicate nominal expansion position as the knob is turned clockwise and the scaffold expands. The knob 16 can be over molded santoprene (or similar material) knob to actuate scaffold at distal tip. The knob 16 can contain grooves for grip. A knob/shaft strain relief 17 can be a shaft strain relief that is integrated into knob. The catheter device of FIG. 13 can further include a handle 19. In some examples, the handle can be split into two mating clamshells (e.g., A half and B half) for assembly. The catheter device of FIG. 13 can further include a ball detent 20. The ball detent 20 (or similar feature) can interact with the base of the knob to produce a click. A slight resistive force at the indicators (for example, “0”, “12”, “14”, “16”, and “18”) on the handle knob. The catheter device of FIG. 13 can include a rotary drive body 21. The interior of over molded handle can contain a spiral drive which can interface with the shaft coupler 22. Turning the knob in the clockwise direction can drive the outer shaft forward. The shaft coupler 22 can be glued to the outer diameter (OD) of the outer shaft and can contain two prongs which extend through the guiding slots 23 and into the spiral recesses of the rotary drive body 21. A shaft coupler guide slots 23 can include two slots in the two mating clamshells of the handle and can prevent rotation of the shaft when the knob is turned, only allowing for the shaft to linearly translate.

In some embodiments, known momentary radial force exerted by scaffold can include, foreshortening basked is paired with a spring in series. The user can dial the knob to a given nominal expansion radius (0 mm, 12 mm, 14 mm, 16 mm, 18 mm). Compression spring is stiffer than scaffold, so scaffold expands first until it contacts the vessel wall. Once vessel wall exerts higher forces on scaffold, compression spring begins to compress. It is assumed that the scaffold also continues to expand as the spring compresses, but that force above the natural expansion of the scaffold is now measured by spring. Wire in shaft translates spring position to sight glass in handle, which shows low/medium/high force.

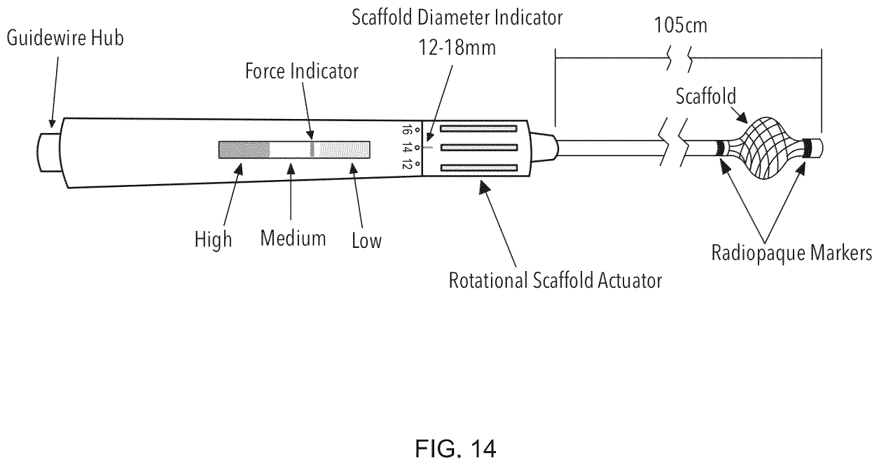

FIG. 14 illustrates an example catheter design.

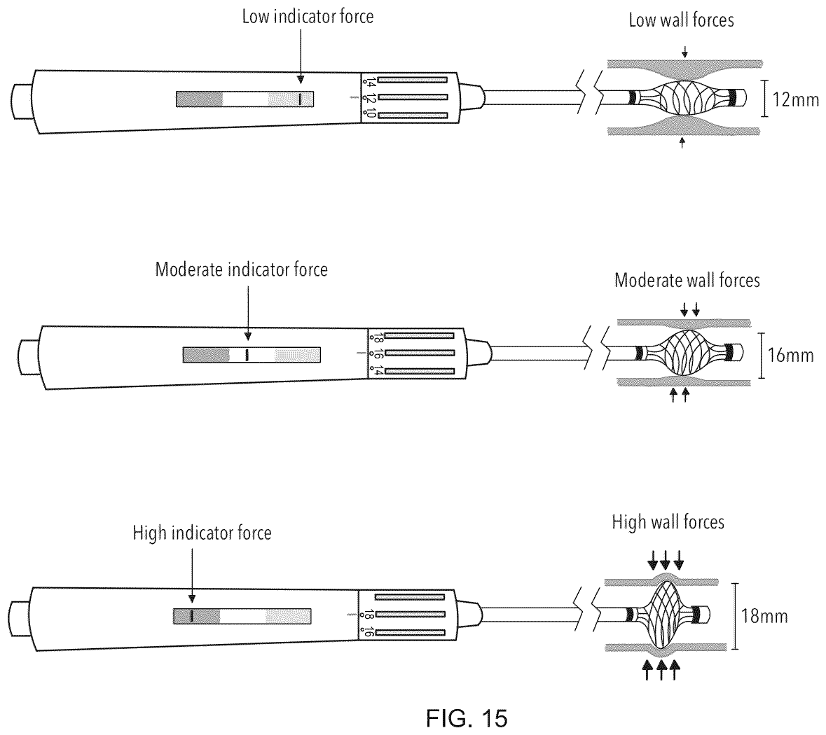

FIG. 15 illustrates the force indicator on the handle of the catheter at various positions when different amount of wall forces are measured (e.g., low, medium, and high).

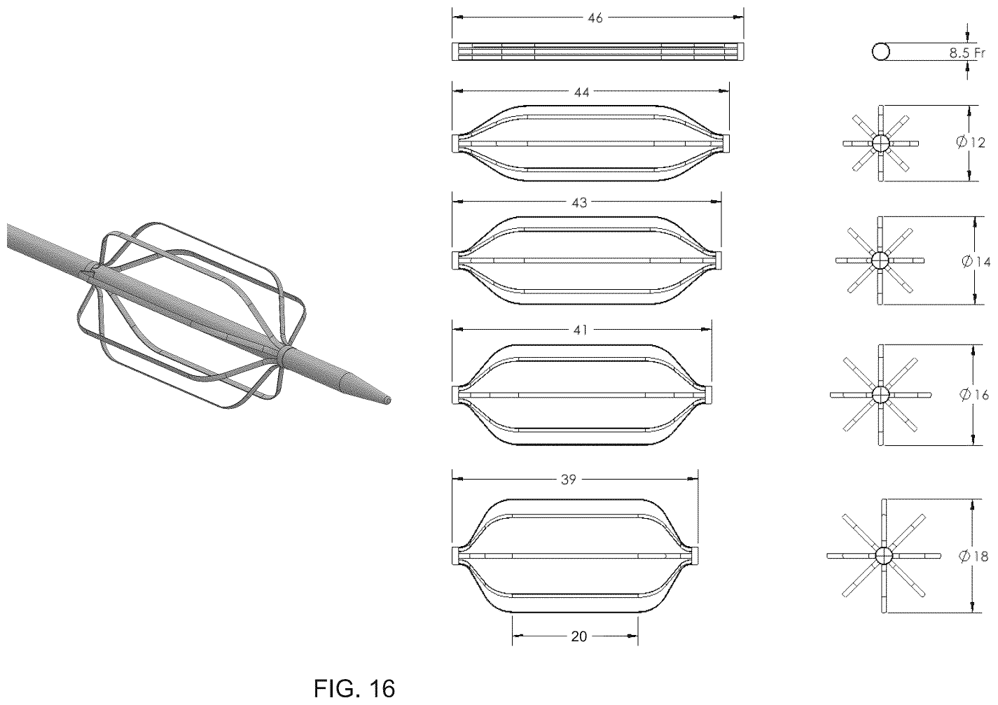

FIG. 16 illustrates an example scaffold design.

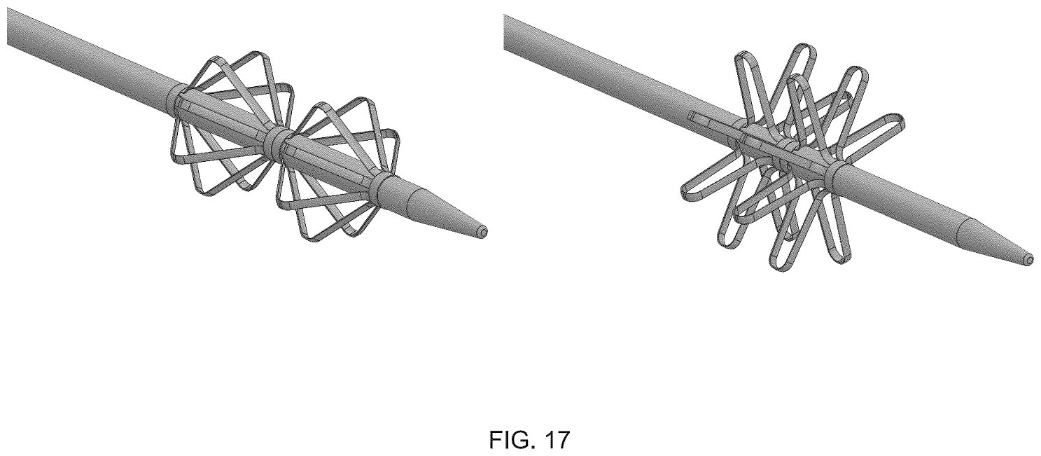

FIG. 17 illustrates an example scaffold design.

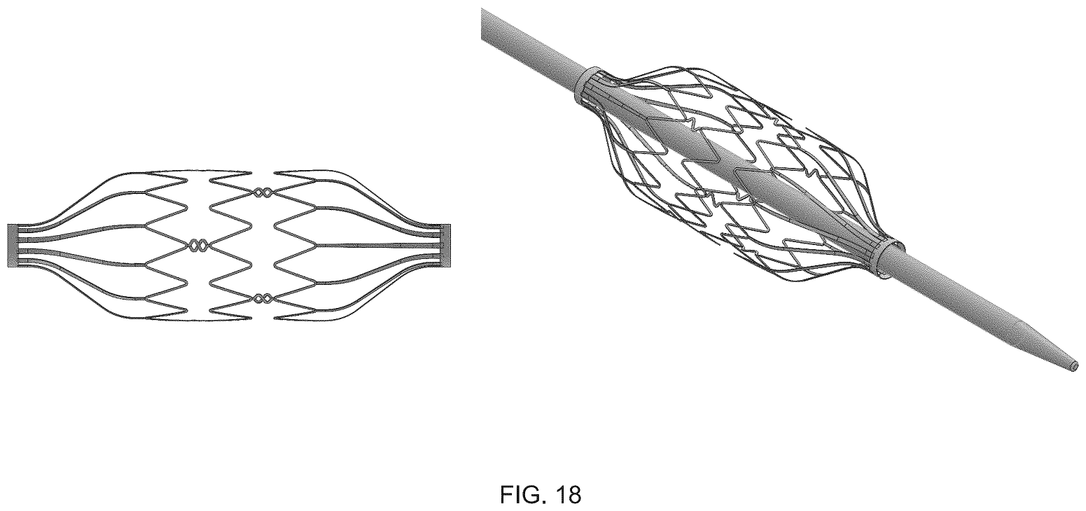

FIG. 18 illustrates an example scaffold design.

### Certain Method Steps

As discussed herein, the device of the present disclosure can be used to address various conditions (e.g., buildup of plaque, wound, tumor, etc.) that has resulted in a narrowed (e.g., compressed) blood vessel. In one example, the following describes some of the non-limiting steps (in any appropriate order) that can be taken to operate a catheter device according to one implementation of the present disclosure. Once a user (e.g., a medical professional) completes the necessary preparatory steps to prepare a subject (e.g., a patient) for the operation, the user can navigate a guidewire (e.g., 035″ guidewire) to the compressed lesion from the left common femoral access or right jugular vein access using the guidewire. The user can then utilize an IVUS catheter to image the compressed iliac vein of the patient. The IVUS results can help the user size the vein diameter. The user can then introduce a fluoroscopic marker (e.g., radiopaque) to the target location and remove the IVUS catheter. The catheter portion 200 of the catheter device 300 can be inserted through an introducer sheath (e.g., a min. 9 French or 9F introducer sheath). Under fluoroscopic imaging, the user can utilize the 0.035″ guidewire to navigate the catheter portion 200 to the target location. Once the catheter portion 200 is at the target location, device activation can be initiated by twisting the knob 130, for example, to the right (i.e., in a clockwise orientation). Such activation causes the outer shaft 120 to move relative to the inner shaft 110, resulting in the expansion of the scaffold 250 and thereby applying a force to the wall of the vessel at the lesion area. The user can utilize catheter feedback (e.g., deployed diameter, compressive force, etc.) to open the vessel and/or determine the proper course of treatment action. For example, the catheter feedback may indicate to a user that the patient does not need a stent and/or the stent strength/size is improper for a particular application. One or more techniques such as fluoroscopy can be used to confirm that the scaffold is opened to the desired width. The aforementioned steps can be repeated as needed to achieve the desired result.

While certain embodiments of the invention have been described, these embodiments have been presented by way of example only and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract, and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. As an example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A catheter device for measuring a force required to radially expand a blood vessel of a subject to an indicated diameter while maintaining blood flow, the device comprising:
a body portion, comprising:
a handle body;
an actuator configured to create a linear motion;
a plurality of sensors located within the handle body, the sensors comprising force sensing and displacement sensing;
a scaffold configured to expand and contract in a transverse direction, the scaffold comprising a plurality of struts, the scaffold configured to be variable in expansion and contraction movement between a collapsed first position and an expanded second position;
a shaft assembly coupled with the body portion, the shaft assembly connecting the body portion to the scaffold and configured to translate force between the body portion and the scaffold, the shaft assembly comprising:
an outer shaft;
an inner shaft within the outer shaft; and
a central lumen defined by a channel in the inner shaft and configured to receive a guidewire;
wherein, the transverse direction is transverse relative to a longitudinal axis of the shaft assembly, and the plurality of sensors are configured to measure force and displacement applied by the blood vessel to the scaffold and translated though the shaft assembly.

2. The catheter device of claim 1, wherein in the collapsed first position, the scaffold includes a first length and a first radial diameter, and in the expanded second position, the scaffold includes a second length and a second radial diameter, the second length shorter than the first length, the second radial diameter larger than the first radial diameter.

3. The catheter device of claim 1, comprising a shuttle coupled to the actuator, the shuttle configured to advance or retract responsive to activating the actuator.

4. The catheter device of claim 3, wherein the shuttle comprises a slot, the displacement sensing comprises a post, the post of the displacement sensing configured to be positioned in the slot of the shuttle.

5. The catheter device of claim 4, wherein the force sensing is positioned between an adapter of the outer shaft and a portion of the shuttle, the force sensing configured to contact the adapter.

6. The catheter device of claim 1, wherein the force sensing comprises a piezoresistive sensor.

7. The catheter device of claim 1, wherein the displacement sensing comprises a slide potentiometer.

8. The catheter device of claim 1, wherein the catheter device is configured such that the scaffold or the shaft assembly does not include a sensor.

9. The catheter device of claim 1, further comprising a digital display configured to provide one or more of: an indication of a diameter of the scaffold, an axial displacement of the shaft assembly, a constrictive wall force of the blood vessel, and/or an axial force on the scaffold.

10. The catheter device of claim 1, wherein each of the plurality of struts comprises branching arms to promote preferential radial expansion of the scaffold while maintaining an apex portion of the scaffold with a profile that is approximately: flat, concave, or convex.

11. The catheter device of claim 1, wherein the actuator is one of a knob, a slide, a gear track, a linear gear train, or a motor.

12. A catheter device for measuring a force required to radially expand a blood vessel of a subject to an indicated diameter while maintaining blood flow, the device comprising:
a body portion, comprising:
a handle body;
an actuator configured to create a linear motion;
a scaffold configured to expand and contract in a transverse direction, the scaffold comprising a plurality of struts, the scaffold configured to be variable in expansion and contraction movement between a collapsed first position and an expanded second position;
a shaft assembly coupled with the body portion, the shaft assembly connecting the body portion to the scaffold and configured to translate force between the body portion and the scaffold, the shaft assembly comprising:
an outer shaft;
an inner shaft within the outer shaft; and
a central lumen defined by a channel in the inner shaft and configured to receive a guidewire;
a plurality of sensors, comprising:
a force sensor positioned in one of:
the shaft assembly;
partially in the shaft assembly and body portion; or
partially in the scaffold, shaft, and body portion;
a displacement sensor positioned in one of:
the shaft assembly;
partially in the shaft assembly and body portion; or
partially in the scaffold, shaft assembly, and body portion;
wherein, the transverse direction is transverse relative to a longitudinal axis of the shaft assembly, and plurality of sensors are configured to measure force and displacement applied by the blood vessel to the scaffold and translated though the shaft.

13. The catheter device of claim 12, wherein the plurality of struts are coupled to the outer shaft at a proximal end of the scaffold and to the inner shaft at a distal end of the scaffold.

14. The catheter device of claim 12, wherein the plurality of struts are coupled to the inner shaft and the outer shaft with a mechanically restraining component.

15. The catheter device of claim 14, wherein the mechanically restraining component comprises a plurality of receiving features configured to mate with the plurality of struts.

16. The catheter device of claim 12, wherein the force sensor and the displacement sensor are communicatively coupled to a processor.

17. The catheter device of claim 16, wherein the processor is configured receiving an analog signal through the shaft assembly, convert the analog signal to a digital signal, and calibrate the digital signal to determine a force required to radially expand a vessel of a subject.

18. The catheter device of claim 12, wherein each of the plurality of struts comprises branching arms to promote preferential radial expansion of the scaffold while maintaining an apex portion of the scaffold with a profile that is approximately: flat, concave, or convex.

19. The catheter device of claim 12, further comprising a digital display configured to provide one or more of: an indication of a diameter of the scaffold, an axial displacement of the shaft assembly, a constrictive wall force of the blood vessel, and/or an axial force on the scaffold.

20. The catheter device of claim 12, wherein the displacement sensor does not include a spring.
